(19) United States
(12) Patent Application Publication
(10) Pub. No.: US 2004/0209360 A1
(43) Pub. Date: Oct. 21, 2004

(54) PVA-based polymer coating for cell culture

(76) Inventors: Steven C. Keith, Chapel Hill, NC (US); Mohammad A. Heidaran, Cary, NC (US); John J. Hemperly, Apex, NC (US); Christopher J. Knors, Raleigh, NC (US)

Correspondence Address: VENABLE LLP, 575 7TH STREET, N.W., WASHINGTON, DC, 20004-1601, US

(21) Appl. No.: 10/418,234

(22) Filed: Apr. 18, 2003

(51) Int. Cl. C12N 5/02; B05D 3/00

(52) U.S. Cl. 435/404; 427/2.11

(57) ABSTRACT

A UV-cross-linkable PVA-based polymer coating for cell culture that provides support for cell adhesion. The polymer coating may also contain bioaffecting molecules reversibly entrapped within the polymer coating that provides necessary nutrients to cell culture. Preferably, the UV-cross-linkable PVA-based polymer is PVA-SbQ.

### FIELD OF THE INVENTION

The present invention relates to a polymer coating for cell culture that promotes cell adhesion. The polymer coating also provides slow release of bioaffecting molecules entrapped within the polymer coating. The present invention also relates to a method for making the polymer coating, which is particularly useful for anchorage-dependent mammalian cell culture.

### BACKGROUND OF THE INVENTION

Cell culture, an important tool for biological research and industrial application, is typically performed by chemically treating the surface of a cell culture device to support cell adhesion and bathing the adherent cells in a culture medium composed of expensive cell growth supplements (i.e., hormones and growth factors).

The phenomenon of “anchorage dependence” provides that anchorage-dependent cells in culture only divide when they are attached to a solid surface but not in liquid suspension. The site of cell adhesion may enable the individual cell to spread out and capture more growth factors and nutrients, to organize its cytoskeleton, and to provide anchorage for the intracellular actin filament and extracellular matrix (ECM) molecules. Thus, a surface that provides sufficient cell adhesion is vital to cell culture and growth.

Further, to support anchorage-dependent mammalian cell growth in cell culture, hormones and protein growth factors, in addition to other cell nutrients, are essential. Often times the source of cell nutrients is serum, the blood-derived fluid that remains after blood has clotted. Serum contains various growth factors for cell growth; however, it is expensive. Protein growth factors are quickly taken up by fast growing cells in cell culture or degraded by proteases in solution. This requires serum containing the growth factors to be replaced every 1-3 days. Failure to replace the serum or provide appropriate nutrients at the appropriate rate will result in arrested cell growth. Mammalian cells deprived of serum or appropriate growth factors stop growing and become arrested, usually between mitosis and S phase, in a quiescent state called G₀. Thus, it is essential to provide the appropriate nutrients at the appropriate rate to cells being cultured.

Efforts have been made toward discovering cell culture that promotes cell adhesion in the absence of serum. Ito et al. (Biotechnol. Prog. 1996, 12, 700-702) disclose that synthesized photoreactive insulin is immobilized onto the wells of polystyrene culture plates by photo-irradiation; and the modification enhances the growth of anchorage-dependent cells. Kobayashi et al. (Biomaterials 1991, Vol. 12 October, 747-751) disclose cell adhesion is promoted by covalently immobilizing cell-adhesive proteins onto the surface of poly(vinyl alcohol) (PVA) hydrogel by diisocyanates, polyisocyanates, and cyanogen bromide. Li et al. (J. Biomater. Sci. Polymer Edn. Vol. 9, No. 3, pp. 239-258 (1998)) disclose PVA foams as scaffolds in hollow fiber membrane-based cell encapsulation devices for cell culture increase catecholamine secretion in dopamine-secreting cells. Kobayashi et al. (Current Eye Research Vol. 10, No. 10, 1991, 899-908) disclose that cell adhesive proteins and molecules covalently immobilized onto PVA hydrogel sheets promote corneal cell adhesion and proliferation.

The PVA hydrogel as disclosed in the references has advantages for cell or tissue culture, such as high water content, softness, bioinertness, and good permeability for cell nutrients including oxygen, glucose, amino acids, lactate, and inorganic ions. However, the material does not allow support for cell adhesion. See for example, Kawase et al., Biol. Pharm. Bull., 22(9), pages 999-1001 (1999). Thus, when cell adhesion is required, especially in anchorage-dependent mammalian cell culture, the properties of the PVA hydrogel become a disadvantage.

### BRIEF SUMMARY OF INVENTION

The present invention provides a poly(vinyl alcohol) (PVA) based hydrogel polymer coating that promotes cell adhesion with no other chemical treatment. The PVA-based hydrogel polymer coating of the present invention also provides sustained release of one or more bioaffecting molecules that are reversibly entrapped within one or more layers of the polymer coating. The polymer coating of the present invention comprises a UV-cross-linkable PVA-based polymer. Preferably, the PVA-based polymer is PVA-(acetalized with N-methyl-4-(p-formyl styryl) pyridinium methosulfate-) (PVA-SbQ).

The PVA-based hydrogel polymer coating of the present invention may also comprise one or more entrapped bioaffecting molecules, which are molecules required for cell viability, cell growth, cell differentiation, or affecting cell adhesion to the culture surface. These bioaffecting molecules may be hormones, growth factors, large molecular weight cell nutrients, molecules capable of cell interaction and cell signaling, DNA molecules capable of being taken up by cells, polysaccharides capable of modulating cell adhesion to the polymer coating, or a combination thereof. The growth factors may be epidermal growth factor, fibroblast growth factor, platelet-derived growth factor, nerve growth factor, transforming growth factor-β, hematopoietic growth factors, interleukins, or any combinations thereof.

The present invention also provides a cell culture system that may contain molecules essential for cell growth at a reduced amount. The cell culture system comprises one or more layers of a PVA-based polymer coating on the surface of a cell culture platform, which can be, among other things, a polystyrene slide or multi-well petri dish. One or more bioaffecting molecules may be reversibly entrapped within the same or different layers of the polymer coating, where they are released into the culture medium and presented to the cells in a controlled manner.

As noted throughout, the polymer coating of the present invention can be either a single layer or multiple layers. A platform can be coated with either a single layer or multiple layers of the polymer coating. Further, the polymer coating (either a single layer or multiple layers) may comprise one or more bioaffecting molecules reversibly entrapped in the same or different layers of the polymer coating.

The present invention also provides a method for making the polymer coating comprising the steps of applying a solution of a UV-cross-linkable PVA-based polymer onto a platform, evenly distributing the solution on the platform, and cross-linking the polymer with ultraviolet light (UV). Preferably, the PVA-based polymer is PVA-SbQ.

Finally, the present invention provides a method for promoting cell adhesion for cell culture by coating a surface of a cell culture platform with a single layer or multiple layers of UV-cross-linkable PVA-based polymer hydrogel coating that may contain one or more bioaffecting molecules reversibly entrapped within the same or different layers of the polymer coating.

### BRIEF DESCRIPTION OF DRAWINGS

FIG. 1 shows the comparison of cell culture on plates coated with UV-cross-linked PVA-based polymer of the present invention (bottom) and plates coated with uncross-linked PVA-based polymer (top).

FIG. 2 shows the comparison of cell culture on a region coated with UV-cross-linked PVA-based polymer of the present invention (right) and uncoated polystyrene region (left).

FIG. 3 shows the comparison of MC3T3-E1 osteoblast cell growth in plates coated with the PVA-based polymer of the present invention, with (“plus”) or without (“minus”) growth factors entrapped within the polymer coating.

### DETAILED DESCRIPTION OF INVENTION

In the present invention, a UV-cross-linkable PVA-based polymer is coated onto the surface of a cell culture device. The UV-cross-linked PVA-based polymer coating is a controllably swellable hydrogel capable of physically entrapping large molecules, such that the physical confinement is capable of reducing diffusional or transportational properties of the large molecules. Upon exposure to aqueous solution, the hydrogel may swell to a desired extent, and thus alter the transport or diffusional properties of the entrapped large molecules, as well as small nutrients from outside environments The diffusional or transportational properties may be controlled by adjustment of the extent of cross-linking. The diffusional or transportational properties of the entrapped large molecule is dependent on the size of the molecule and the extent of the cross-linking. Optimization of the desired diffusional or transportational properties is achieved by routine experimentation.

Preferably, the PVA-based polymer is PVA-(acetalized with N-methyl-4-(p-formyl styryl) pyridinium methosulfate-) (PVA-SbQ). The amount of SbQ attached to the PVA can vary from about 0.5 mol % to about 10 mol %. Variants of the SbQ moiety exist to provide for use of different wavelength for cross-linking, ranging from about 350 nm to about 600 nm. The more SbQ content in the PVA-based polymer, the faster the UV cure and the greater the cross-linking density of the resultant polymer coating. A higher degree of cross-linking is desired in the polymer coating of the present invention so that the resultant polymer coating is more insoluble in water and culture medium.

Suitable PVA-SbQ polymers used in the present invention are preferably free of antimicrobial agents and have neutral pH. (Antimicrobials are added to most PVA-SbQ formulations to improve the shelf life.) An example of a suitable PVA-SbQ is the PVA-based polymer designated SPP-LS-400, which is manufactured by Charkit (Darian, Conn.). In this particular PVA-SbQ sample, no antimicrobial agents are present. The characteristics of the polymer are: the degree of polymerization (DP) is 500; the degree of saponification (DS) is 88%; the SbQ content in molar percentage is 4.1±0.15; the solid content is 13.3%; the pH of the polymer is 5.5˜7; and the viscosity at 25° C. is 2000±500 cp.

The polymer coating of the present invention may be applied to any surface except TEFLON. These surfaces include glass or any gas plasma-treated polystyrene. The surface may be, among other things, a polystyrene slide, as well as a single-well or multi-well petri dish.

The polymer coating may be applied to the surface using a variety of mechanisms, including, as an example and not a limitation, spinning, foaming, and dipping. More specifically, the solution containing the PVA-based polymer is applied to a surface using any mechanisms provided that it is evenly distributed and the depth does not prevent cross-linking by ultraviolet light (UV).

The UV-cross-linked PVA-based polymer coating of the present invention provides enhanced cell adhesion even in the absence of adhesion-promoting molecules attached to the surfaces. In comparison, untreated surface do not support cell adhesion, and the non-cross-linked PVA polymer coating processed in similar manners supports very low levels of protein adsorption and cell adhesion over time. Such non-cross-linked PVAs must be coated with collagen, fibronectin, or RGD peptides to support cell adhesion for cell culture. Enhanced cell adhesion on cross-linked PVA surfaces may be associated with the multiplicity of hydroxyl groups or the result of cross-linking of these hydroxyl groups.

The polymer coating may also contain entrapped bioaffecting molecules, which are molecules required for cell viability, cell growth, cell differentiation, or affecting cell adhesion to the culture surface. These bioaffecting molecules may be hormones, growth factors, large molecular weight cell nutrients, molecules capable of cell interaction and cell signaling, DNA molecules capable of being taken up by cells, polysaccharides capable of modulating cell adhesion to the polymer coating, or a combination thereof. The growth factors may be epidermal growth factor, fibroblast growth factor, platelet-derived growth factor, nerve growth factor, transforming growth factor-β, hematopoietic growth factors, interleukins, or any combinations thereof. Large molecular weight cell nutrients may include, for example, protein nutrients that are beneficial for certain types of mammalian cell culture.

One or more bioaffecting molecules reversibly entrapped in the polymer coating (either single or multiple layers) of the present invention may be controllably released into the culture medium. The sustained release of the bioaffecting molecules provides an effective and better presentation of the molecules to the cells. Cells respond well to the relatively low levels of the bioaffecting molecules controllably released from the polymer coating. The bioaffecting molecules exert their effects on cells cultured on the coating for over 1-3 days and the effects last over 3-7 days. The release rate of the entrapped molecule may be optimally controlled by the loading amount of the bioaffecting molecules, the thickness of the polymer coating, the size of the bioaffecting molecules, and the density of cross-linking. Since the bioaffecting molecules are released over time, the culture medium need not be as frequently replaced as in normal cell culture. As a result, less serum-containing medium and fewer growth factors are required for cell culture.

Long-chain polymers, in general, offer a diffusion barrier to molecules such as growth factors as the chains are more or less closely spaced and form pore spaces in between. As the polymer chains are made more resistant to movement by an increase in molecular mass or bonding to other chains, the diffusion barrier increases. Such effects are achieved by cross-linking. Thus, the UV-induced cross-linking within the PVA-SbQ of the present invention gives the polymer a more rigid backbone than what is normally obtained in PVA polymer. Uncross-linked PVA polymers re-dissolve in solution, while cross-linked PVA-SbQ is relatively un-dissolvable in solution. The cross-linked polymer provides a larger diffusion barrier to bioaffecting molecules, such as the growth factors, than the non-cross-linked polymer, because the intra- and intermolecular spacing between the polymer chains is reduced thus effecting the diffusion of bioaffecting molecules.

When the cross-linking density is adjusted by the content of the SbQ moiety in the PVA-based polymer or the conditions for cross-linking such as time and wavelength, one may control the diffusion rate of the entrapped bioaffecting molecules. By selecting the proper weight percentage of the PVA polymer in solution and the molar percentage of the attached SbQ moiety of the PVA-based polymer, one can change either or both the film thickness and the cross-linking density, and thus, alter and tailor the diffusion properties of the entrapped material for needs of slow-release and controlled-release. The water of the polymer coating enables improved distribution and dispersion of the bioaffecting molecules within the polymer coating.

The initial thickness of the polymeric coating can be varied, and because of the swelling property of the hydrogel in solution, it is difficult to predict the final thickness of the polymer coating in culture medium. It is noted, however, that the coating thickness can correlate with the duration of the sustained release effect. The thickness is also dependent on the degree and completeness of cross-linking desired. If the polymer hydrogel coating is too thick, the film cannot be cross-linked by UV light completely, because the exposed surface shields the interior from the UV light source. The polymer hydrogel coating having thickness of up to 10 microns has been successfully cross-linked under the UV light.

The present invention provides a simplified cell culture system that contains the UV-cross-linked PVA-based polymer coating on a platform surface that not only supports cell adhesion without chemical treatment, but also has sustained release and supplement of various cell nutrients, including growth factors and large molecular weight nutrients for cell growth. The slow release of molecules provides a system that requires less manual manipulation, and fewer nutrients less frequently. Thus, the cell culture system of the present invention is convenient and cost saving. As an example, the UV-cross-linked PVA-based polymer coating on a surface by the method of the present invention is produced by:

1. Making the PVA-based Solution.

In general, the native PVA-based polymer is diluted and dissolved in water to make the solution. The degree of dilution depends on the desired thickness of the coated polymer layer. One of ordinary skill in the art would make films from formulations with a wide range of DP, DS, SbQ content, solids content, and final dilution.

Preferably, the PVA-SbQ polymer, which is commercially available in a 13% solution (w/v (13 grams of PVA-SbQ dissolved in 100 ml solution)) is diluted in water to obtain a PVA-SbQ polymer solution of about 1˜13% (w/v). Preferably, the PVA-SbQ polymer solution has a concentration of about 2˜13% (w/v). More preferably, the PVA-SbQ polymer solution has a concentration of about 2.6˜7% (w/v). The undiluted PVA-SbQ polymer solution having 13% (w/v), as compared with more diluted solution, has higher viscosity, yet it is possible to use the solution in the present invention.

The bioaffecting molecules may also be added to the solution and dispersed or dissolved in the solution with the PVA-based polymer. These molecules preferably are large enough (greater than or equal to about 5,000 Daltons) so that they may be physically entrapped within the PVA-based polymer coating after cross-linking. There is no specific requirement for the dissolving conditions of the bioaffecting molecules as long as they remain stable in the solution. Thus, suspensions, emulsions, nanoparticles, microparticles, and the like, of bioaffecting molecules in combination with the UV-cross-linkable PVA are within the scope of the instant invention. The concentration of the bioaffecting molecules can be as minimum as that having a biological effect on the cultured cells and as maximum as that soluble in the solution containing the PVA-based polymer. As noted above, even high concentrations of bioaffecting molecules that lead to insoluble aggregation may also be useful if they dissolve slowly to release the molecules. Preferably, the concentration of the bioaffecting molecule is about 0.01 ng/ml to 3000 ng/ml.

2. Applying the Solution on the Platform.

The mixed solution is applied on the surface of the platform for cell culture using any mechanisms provided that it tends to be evenly distributed. For example, the solution can be cast or sprayed on the surface, or the platform can be dipped in the solution so that the solution is evenly distributed. The platform can be a variety of surfaces, including glass, a polystyrene slide or a multi-well petri dish.

3. Distributing the Solution Evenly on the Platform Surface.

The platform surface applied with the solution is spun so that the solution is evenly distributed on the surface and forms a coating of a desired thickness. The spinning-coating process is well known for making film coating; however, other coating process may be used to apply the polymer coating of the present invention to a substrate. When the solution is cast onto the substrate and the substrate is spun at high speed, centripetal acceleration will cause most of the solution to spread to, and eventually off, the edge of the substrate, leaving a thin layer of film on the surface. The final coating thickness and other properties depend on the nature of the solution (viscosity, drying rate, percent solids, surface tension) and the parameters chosen for the spin process. One skilled in the art is able to adjust the thickness of the film such as by varying the either or both of the volume or concentration of the spinning solution and the spinning speed. Generally, the spinning speed is equal to or above about 1000 rpm. The upper limit of the spinning speed is strictly set by the equipment available and up to about 8000 rpm. Preferably, the spinning speed is about 3000 rpm and time is about 1 minute. A suitable spincoater, for example, is Spincoater Model P6700 from Specialty Coating System, Inc., 5707 W Minnesota St., Indianapolis, Ind. 46241.

4. UV-Cross-Linking.

The surface of the platform coated with the solution of the PVA-based polymer is treated with UV light for cross-linking reaction. The SbQ moiety can be selected to cross-link at a particular wavelength of light, preferably such wavelength is that which minimized photoinduced damage to the bioaffecting molecule or provides manufacturability benefit. One of ordinary skill in the art would be able to select the appropriate SbQ moiety, wavelength of light, and time for UV irradiation depending on the thickness of the coating and the desired degree of completeness of cross-linking. Variants of the SbQ moiety exist to provide for use of different wavelength for cross-linking, ranging from about 350 nm to about 600 nm. The UV cross-linking reaction takes from about 5 seconds to 20 minutes, and preferably, about 10 seconds to 10 minutes.

5. Multiple Layers of Coating.

The platform may be further coated with another layer or multiple layers of the cross-linked PVA-based polymer by the same spin-coating process (or any coating process suitable to the artisan skilled in the art), and the same or different bioaffecting molecules or combinations thereof may be entrapped in the same or different layers of the polymer coating, thus creating a variety of microenvironments optimized for various cell growth and cell culture.

The following examples are illustrative only and are not intended to limit the scope of the present invention. Reasonable variations, such as those that occur to the reasonable artisan, can be made without departing from the scope of the present invention.

### EXAMPLE 1

### Making the Polymer Coating of the Present Invention without Bioaffecting Molecules

UV-cross-linkable PVA-SbQ was dissolved in water to make a solution of about 7% (w/v). 100 μl of the solution was cast onto polystyrene petri dishes and spun at about 3000 rpm for 60 seconds so that a polymeric film having a thickness of about 1 micron was uniformly spread onto the polystyrene surface. The film was cross-linked under a 450 W UV light for 10 seconds.

### EXAMPLE 2

### Making the Polymer Coating of the Present Invention with Bioaffecting Molecules

Following the method of Example 1, platelet-derived growth factor-B (PDGF-B) (4 μl of 200 μg/ml solution) and insulin (10 μl of 50 μg/ml solution) were added to and dispersed in 2 ml of 7% PVA-SbQ solution. (The final concentrations of PDGF-B and insulin were 0.4 ng/ml and 250 ng/ml, respectively.) Then, the solution was cast on the bottom of petri dishes. A thin film of the polymer hydrogel was prepared on the bottom of the 60 mm diameter petri dish by spin-coating with 100 μl PVA-SbQ solution containing growth factors at 3000 rpm for 60 seconds so that a polymeric film having a thickness of about 1 micron was uniformly spread onto the polystyrene surface. The films were cross-linked by UV irradiation for 10 minutes in a UV light box from 3D Systems (model PCA250, 10 UV bulbs, each 40 W at 420 nm).

### EXAMPLE 3

### Cell Culture Comparison on Uncoated Plates PVA Coated Uncross-linked Plates and PVA Coated Cross-linked Plates

Uncross-linked plates were prepared by spin-coating an aqueous solution of PVA (5% w/v) without the SbQ moiety onto 60 mm diameter petri dishes at 3000 rpm for 60 seconds. The plates were allowed to dry at room temperature.

Cross-linked plates were prepared by spin coating. Each petri dish (60 mm diameter) was coated with 100 μl PVA-SbQ solution (5% w/v) at 3000 rpm for 60 seconds. The plates were allowed to dry at room temperature. The plates were cross-linked with UV light for 1 minute.

UV-cross-linked and uncross-linked PVA-based polymer coated plates were used for cell culture of human prostate tumor cell line PC-3 cells for 5 days. After 5 days, the plates were washed, fixed with formalin, and stained with haematoxylin and eosin.

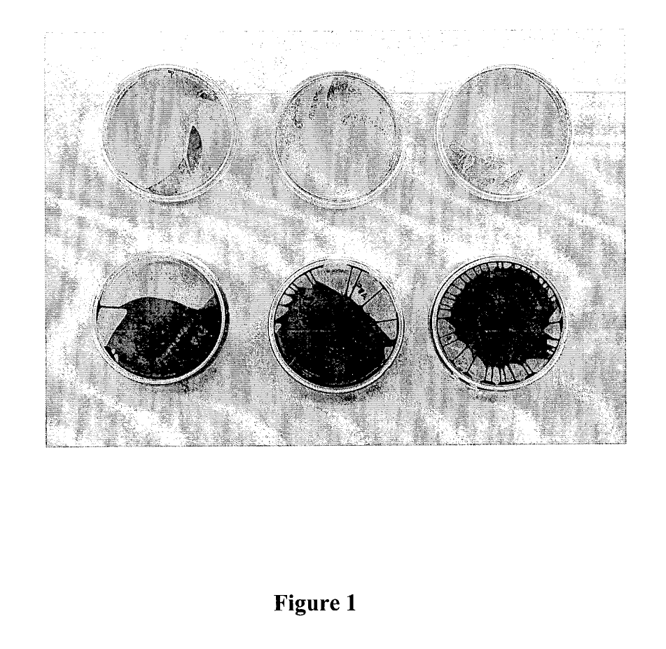

As shown in FIG. 1, uncross-linked dishes (at the top) did not support cell adhesion. No staining was observed on the uncross-linked dishes. As shown in the bottom of FIG. 1, the UV cross-linked PVA-SbQ coated plates did support cell attachment (as indicated by dark staining). On these UV cross-linked PVA-SbQ coated plates, there were regions that were not coated with the polymer (probably due to the viscosity of the solution or amount of the solution used or spin speed). These uncoated regions formed when the petri dish was spun and tracks of the solution extended radially towards the outer edge of the petri dish, leaving the uncoated regions therebetween. As seen in FIG. 1 at the bottom, these uncoated regions (clear staining) flanking the coated and stained regions on the petri dish were not stained and did not support cell adhesion. Higher magnification of the figure (not shown here) showed that cells attached to the coated surface and spread themselves for optimal cell growth.

### EXAMPLE 4

### Cell Culture on the Polymer Coated Plates and MC3T3-E1 Cell Attachment and Growth

Mouse MC3T3-E1 cells (1×10⁵) in 4 ml BITS medium (BSA, insulin, transferrin, and selenium) were added to dishes each coated with 100 μl solution of UV-cross-linked PVA-SbQ containing insulin and platelet-derived growth factor as prepared in examples 1 and 2.

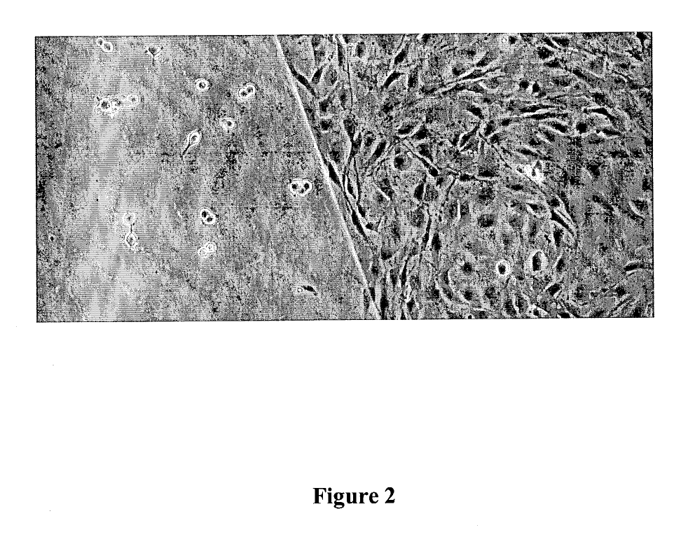

As indicated in FIG. 2, on the left side of the photomicrograph, the uncoated region of the plate did not support the attachment and growth of mouse MC3T3-E1 osteoblasts. There were a few cells seen on the uncoated region (left side of the figure), however, the morphology of the cells differed substantially from the ones on the right. The cells on the uncoated regions did not form attachment to the substrate surface and did not spread on the surface. No cell growth was observed in the uncoated region. On the right side, which was an area of cross-linked PVA-SbQ containing a mixture of insulin and platelet-derived growth factor, the cells attach, spread, and proliferate. The spread cells were many micrometers in diameter. Cells did adhere well to PVA-SbQ coated plates, indicating that the PVA-SbQ coating support cell attachment of a variety of cell types.

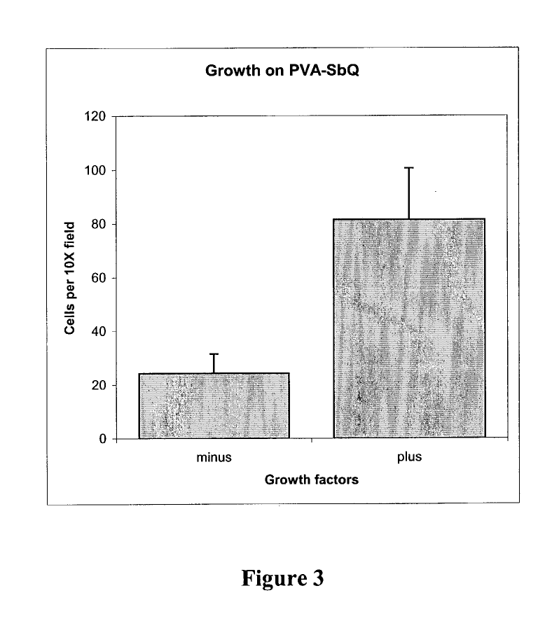

Moreover, cell growth was enhanced on the plates containing insulin and platelet-derived growth factor as compared with the plates coated with UV cross-linked PVA-SbQ that did not contain growth factors after 5 days in culture. As indicated in FIG. 3, the numbers of cells per 10× field were 82±19 for growth factors-containing coated dish and 24±7 for non-growth factor-containing dish. The difference was statistically significant (P=0.002). Therefore, the UV-cross-linked PVA-SbQ coated dish containing entrapped growth factors showed best results in supporting cell adhesion and cell growth.

We claim:

1. A polymer coating for cell culture comprising a UV-cross-linkable hydrogel of a polyvinyl alcohol (PVA)-based polymer wherein the hydrogel has been cross-linked and the PVA-based polymer coating provides for cell adherence.

2. The polymer coating of claim 1, further comprising one or more bioaffecting molecules reversibly entrapped within the PVA-based polymer coating.

3. The polymer coating of claim 1, wherein the PVA-based polymer is PVA-(acetalized with N-methyl-4-(p-formyl styryl) pyridinium methosulfate) (PVA-SbQ).

4. The polymer coating of claim 3, wherein the SbQ moiety in the PVA-SbQ is 0.5 to 10 mol %.

5. The polymer coating of claim 1, wherein thickness of the coating is no more than 10 microns.

6. The polymer coating of claim 2, wherein the bioaffecting molecules are selected from the group consisting of hormones, growth factors, large molecular weight cell nutrients, molecules capable of cell interaction and cell signaling, DNA molecules capable of being taken up by cells, polysaccharides capable of modulating cell adhesion to the polymer coating, and combinations thereof.

7. The polymer coating of claim 6, wherein the growth factors are selected from the group consisting of epidermal growth factor, fibroblast growth factor, platelet-derived growth factor, nerve growth factor, transforming growth factor-β, hematopoietic growth factors, interleukins, and combinations thereof.

8. The polymer coating of claim 2, wherein the concentration of bioaffecting molecules in the polymer coating is 0.01 ng/ml to 3000 ng/ml.

9. The polymer coating of claim 1, comprising multiple layers of the PVA-based polymer coating.

10. The polymer coating of claim 2, comprising multiple layers of the PVA-based polymer coating and one or more bioaffecting molecules reversibly entrapped in the same or different layers of the PVA-based polymer coating.

11. The polymer coating of claim 2, wherein the bioaffecting molecules reversibly entrapped within the PVA-based polymer coating are released from the polymer coating to cell culture over time.

12. The polymer coating of claim 3, wherein the PVA-SbQ is free of antimicrobial agents.

13. A cell culture system comprising:
one or more layers of the polymer coating of claim 1 on a surface of a platform for cell culture.

14. The cell culture system of claim 13, further comprising cell culture medium containing serum at a reduced amount.

15. The cell culture system of claim 13, further comprising serum-free cell culture medium.

16. The cell culture system of claim 13, wherein one or more of the layers of the polymer coating comprises one or more bioaffecting molecules reversibly entrapped therein.

17. The cell culture system of claim 16, wherein the bioaffecting molecules are selected from the group consisting of hormones, growth factors, large molecular weight cell nutrients, molecules capable of cell interaction and cell signaling, DNA molecules capable of being taken up by cells, polysaccharides capable of modulating cell adhesion to the polymer coating, and combinations thereof.

18. The cell culture system of claim 16, wherein one or more of the bioaffecting molecules are entrapped in the same layer or different layers of the polymer coating.

19. A method for making a polymer coating comprising one or more layers comprising:
applying a layer of said polymer coating from a solution containing a UV-cross-linkable PVA-based polymer onto a platform surface;
distributing a thickness of said polymer coating on the surface;
cross-linking the PVA-based polymer on the platform surface with UV light; and
repeating said casting, distributing and cross-linking for each layer.

20. The method of claim 19, wherein the cross-linkable PVA-based polymer is PVA-SbQ.

21. The method of claim 21, wherein the SbQ moiety in the PVA-SbQ polymer is 0.5 to 10 mol %.

22. The method of claim 19, wherein the solution containing the cross-linkable PVA-based polymer has a concentration of 1-13% (w/v).

23. The method of claim 19, wherein the solution containing the cross-linkable PVA-based polymer has a concentration of 13% (w/v).

24. The method of claim 19, wherein the solution containing the cross-linkable PVA-based polymer has a concentration of 2.6-7% (w/v).

25. The method of claim 19, wherein the platform is spun at 1000 to 8000 rpm.

26. The method of claim 19, wherein the platform is spun at 3000 rpm.

27. The method of claim 19, wherein the solution of the UV-cross-linkable PVA-based polymer further comprises bioaffecting molecules.

28. The method of claim 27, wherein the bioaffecting molecules are selected from the group consisting of hormones, growth factors, large molecular weight cell nutrients, molecules capable of cell interaction and cell signaling, DNA molecules capable of being taken up by cells, polysaccharides capable of modulating cell adhesion to the polymer coating, and combinations thereof.

29. The method of claim 19, wherein the cross-linking reaction takes place for 5 seconds to 20 minutes.

30. The method of claim 19, wherein the cross-linking reaction takes place for 10 seconds to 10 minutes.

31. The method of claim 19, wherein the cross-linking reaction takes place at a wavelength of 350 nm to 600 nm.

32. The method of claim 19, wherein the surface is selected from the group consisting of glass, a polystyrene slide and a multi-well petri dish

33. A polymer coating made by the process of claim 19.

34. A method for enhancing cell adhesion comprising:
coating a platform surface for cell culture with a UV-cross-linkable PVA-based polymer; and
cross-linking the coated PVA-based polymer with UV light.

35. A method for sustained release of bioaffecting molecules to cell culture comprising:
coating a platform for cell culture with one or more layers of a UV-cross-linkable PVA-based polymer hydrogel solution having one or more bioaffecting molecules reversibly entrapped therein;
cross-linking each layer of the hydrogel with UV light to form a polymer coating;
adding cell culture medium to the polymer coating; and
allowing the bioaffecting molecules to be released.

36. The polymer coating of claim 35, wherein the bioaffecting molecules are selected from the group consisting of hormones, growth factors, large molecular weight cell nutrients, molecules capable of cell interaction and cell signaling, DNA molecules capable of being taken up by cells, polysaccharides capable of modulating cell adhesion to the polymer coating, and combinations thereof.

37. The polymer coating of claim 36, wherein the growth factors are selected from the group consisting of epidermal growth factor, fibroblast growth factor, platelet-derived growth factor, nerve growth factor, transforming growth factor-β, hematopoietic growth factors, interleukins, and combinations thereof.
